(19) United States
(12) Patent Application Publication
(10) Pub. No.: US 2006/0142239 A1
(43) Pub. Date: Jun. 29, 2006

(54) Potassium or sodium salt of (-)-2-(4-hydroxyphenyl)ethyl!-thio-3-'4-{4-'(methzlsulfonzl)oxy !phenoxy}phenyl!propanoic acid and their use in medicine

(76) Inventor: Anderson Joseph Ryan, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 10/561,183

(22) PCT Filed: Jun. 18, 2004
(86) PCT No.: PCT/GB04/02624

§ 371 Date: Dec. 16, 2005

(30) Foreign Application Priority Data
Jun. 18, 2003 (GB) 0314097.7
Jul. 10, 2003 (GB) 0316181.7

(51) Int. Cl. A61K 31/7072; A61K 31/66; A61K 31/513

(52) U.S. Cl. 514/49; 514/114; 514/269

(57) ABSTRACT

A method for the production of a vascular damaging effect in a warm-blooded animal such as a human which is optionally being treated with ionising radiation, particularly a method for the treatment of a cancer involving a solid tumour, such as colorectal cancer, which comprises one of: the administration of ZD6126 in combination with 5-FU; the administration of ZD6126 in combination with CIT-11; and the administration of ZD6126 in combination with 5-FU and CPT-11. Also claimed are pharmaceutical compositions and kits comprising one of ZD6126 and CPT-11; and ZD6126 and 5-FU and CPT-11. Further claimed is use of one of: ZD6126 and 5-FU; ZD6126 and CPT-11; and ZD6126 and 5-FU and CPT-11, in the manufacture of a medicament for use in the production of a vascular damaging effect in a warm-blooded animal which is optionally being treated with ionising radiation.

The present invention relates to a method for the production of a vascular damaging effect in a warm-blooded animal such as a human which is optionally being treated with ionising radiation, particularly a method for the treatment of a cancer involving a solid tumour, which comprises one of: the administration of ZD6126 in combination with 5-FU; the administration of ZD6126 in combination with CPT-11; and the administration of ZD6126 in combination with 5-FU and CPT-11; to a pharmaceutical composition comprising one of: ZD6126 and 5-FU; ZD6126 and CPT-l1; and ZD6126 and 5-FU and CPT-11; to a combination product comprising one of: ZD6126 and 5-FU; ZD6126 and CPT-11; and ZD6126 and 5-FU and CPT-11, for use in a method of treatment of a human or animal body by therapy; to a kit comprising one of: ZD6126 and 5-FU; ZD6126 and CPT-11; and ZD6126 and 5-FU and CPT-11; to the use of one of: ZD6126 and 5-FU; ZD6126 and CPT-11; and ZD6126 and 5-FU and CPT-11, in the manufacture of a medicament for use in the production of a vascular damaging effect in a warm-blooded animal such as a human which is optionally being treated with ionising radiation.

In the USA, in 1999, it was estimated that there would be 129,400 new cases of CRC (colorectal cancer), accounting for 11% of all new cases of cancer (LandisS H, et al. CA Cancer J Clin 1999; 49: 8-31). CRC was the third most common cancer in men, after lung and prostate cancer, estimated to account for approximately 27,900 deaths, and the third most common in females, after lung and breast cancer, estimated to account for approximately 28,600 deaths in 1998.

Prognosis for patients with CRC is dependent on the disease stage at diagnosis. Results from a study by Sinicrope et al. (Gastroenterology 1995; 109: 984-993), which examined outcome following treatment with surgery versus stage of disease at diagnosis, demonstrated that there was a significant decline in survival in patients diagnosed with advanced disease compared with those who were diagnosed early. The 5-year survival rate for patients with localised disease is approximately 90% compared with <10% for those patients with distant metastases, such as liver and lung (Landis S H, et al. CA Cancer J Clin 1998; 48: 6-29).

Surgery is the main treatment for advanced CRC and is particularly successful in patients with advanced disease characterised by isolated resectable primary tumour and metastases. Radiotherapy may also be used in some cases to treat CRC, although radiotherapy is rarely used alone. In most patients with advanced disease not amenable to surgery, palliative chemotherapy is the most appropriate treatment option. The past decade has seen a number of advances in the treatment options for advanced CRC compared with the previous 40 years. In the 1950s, the only available option was palliative surgery to improve quality of life and prevent bowel obstruction. In 1957, Heidelberger et al (Nature 1957; 179: 663-666) first demonstrated the antitumour effects 5-FU in rodents. A 5-year study, published in 1962 by Ansfield et al (JAMA 1962; 181: 295-299) on the use of 5-FU in patients with a variety of tumours, demonstrated its effectiveness in patients with cancer of the colon and rectum. In the 1980s, modulating agents, such as leucovorin, came into widespread use as they were shown to potentiate the activity of 5-FU (Machover D, et al. Cancer Treat Rep 1982; 66: 1803-1807).

Several new agents were introduced for the treatment of advanced CRC in the late 1990s, including irinotecan (CPT-11, Campto™, Camptosar™), a topoisomerase inhibitor (Bleiberg H. Anti-Cancer Drugs 1998; 9: 18-28). Recently, new oral 5-FU agents including capecitabine (Xeloda™) and uracil/tegafur (UFT) have been developed. There remains however, the need for alternative treatments for cancers such as CRC.

Normal angiogenesis plays an important role in a variety of processes including embryonic development, wound healing and several components of female reproductive function. Undesirable or pathological angiogenesis has been associated with disease states including diabetic retinopathy, psoriasis, cancer, rheumatoid arthritis, atheroma, Kaposi's sarcoma and haemangioma (Fan et al, 1995, Trends Pharmacol. Sci. 16: 57-66; Folkman, 1995, Nature Medicine 1: 27-31). Formation of new vasculature by angiogenesis is a key pathological feature of several diseases (J. Folkman, New England Journal of Medicine 333, 1757-1763 (1995)). For example, for a solid tumour to grow it must develop its own blood supply upon which it depends critically for the provision of oxygen and nutrients; if this blood supply is mechanically shut off the tumour undergoes necrotic death. Neovascularisation is also a clinical feature of skin lesions in psoriasis, of the invasive pannus in the joints of rheumatoid arthritis patients and of atherosclerotic plaques. Retinal neovascularisation is pathological in macular degeneration and in diabetic retinopathy.

Reversal of neovascularisation by damaging the newly-formed vascular endothelium is expected to have a beneficial therapeutic effect. International Patent Application Publication No. WO 99/02166 describes tricyclic compounds that surprisingly have a selective damaging effect on newly formed vasculature as compared to the normal, established vascular endothelium of the host species. This is a property of value in the treatment of disease states associated with angiogenesis such as cancer, diabetes, psoriasis, rheumatoid arthritis, Kaposi's sarcoma, haemangioma, lymphoedema, acute and chronic nephropathies, atheroma, arterial restenosis, autoimmune diseases, acute inflammation, excessive scar formation and adhesions, endometriosis, dysfunctional uterine bleeding and ocular diseases with retinal vessel proliferation including macular degeneration.

Compounds which damage newly formed vasculature are vascular targeting agents (VTAs) and are also known as vascular damaging agents (VDAs).

One such compound described in International Patent Application Publication No. WO 99/02166 is N-acetylcolchinol-O-phosphate, (also know as (5S)-5-(acetylamino)-9,10,11-trimethoxy-6,7-dihydro-5H-dibenzo[a,c]cyclohepten-3-yl dihydrogen phosphate; Example 1 of WO 99/02166), which is referred to herein as ZD6126:

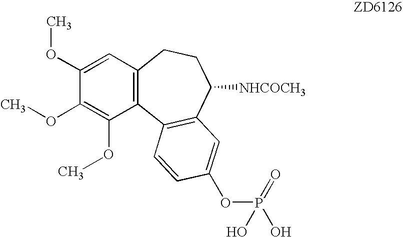

It is believed, though this is not limiting on the invention, that ZD6126 damages newly-formed vasculature, for example the vasculature of tumours, thus effectively reversing the process of angiogenesis. It has been reported that ZD6126 selectively disrupts tumour vasculature leading to vessel occlusion and extensive tumour necrosis (Davis P D, Hill S A, Galbraith S M, et al. Proc. Am. Assoc. Cancer Res. 2000; 41: 329).

In WO 99/02166 it is stated that: “compounds of the invention may be administered as sole therapy or in combination with other treatments. For the treatment of solid tumours compounds of the invention may be administered in combination with radiotherapy or in combination with other anti-tumour substances for example those selected from mitotic inhibitors, for example vinblastine, paclitaxel and docetaxel; alkylating agents, for example cisplatin, carboplatin and cyclophosphamide, antimetabolites, for example 5-fluorouracil, cytosine arabinoside and hydroxyurea; intercalating agents for example adriamycin and bleomycin; enzymes, for example asparaginase; topoisomerase inhibitors for example etoposide, topotecan and irinotecan; thymidylate synthase inhibitors for example raltitrexed; biological response modifers for example interferon; antibodies for example edrecolomab, and anti-hormones for example tamoxifen. Such combination treatment may involve simultaneous or sequential application of the individual components of the treatment.”

Nowhere in WO 99/02166 is one of the specific combinations of ZD6126 and 5-FU; ZD6126 and CPT-11; and ZD6126 and 5-FU and CPT-11, suggested.

Nowhere in WO 99/02166 does it state that use of any compound of the invention therein with other treatments will produce surprisingly beneficial effects.

Unexpectedly and surprisingly we have now found that the particular compound ZD6126 used in combination with a particular selection of combination therapies, namely with one of: 5-FU; CPT-11; and 5-FU and CPT-11, produces significantly better vascular damaging effects than any one of: ZD6126; 5-FU; CPT-11; and 5-FU and CPT-11 used alone. According to one aspect of the present invention, ZD6126 used in combination with one of: 5-FU; CPT-11; and 5-FU and CPT-11 produces significantly better anti-cancer effects than any one of: ZD6126; 5-FU; CPT-11; and 5-FU and CPT-11 used alone. According to one aspect of the present invention, ZD6126 used in combination with one of: 5-FU; CPT-11; and 5-FU and CPT-11 produces significantly better effects on solid tumours than any one of: ZD6126; 5-FU; CPT-11; and 5-FU and CPT-11 used alone. According to one aspect of the present invention, ZD6126 used in combination with one of: 5-FU; CPT-11; and 5-FU and CPT-11 produces significantly better effects in colorectal cancer than any one of: ZD6126; 5-FU; CPT-11; and 5-FU and CPT-11 used alone.

Anti-cancer effects of a method of treatment of the present invention include, but are not limited to, anti-tumour effects, the response rate, the time to disease progression and the survival rate. Anti-tumour effects of a method of treatment of the present invention include, but are not limited to, inhibition of tumour growth, tumour growth delay, regression of tumour, shrinkage of tumour, increased time to regrowth of tumour on cessation of treatment, slowing of disease progression. It is expected that when a method of treatment of the present invention is administered to a warm-blooded animal such as a human, in need of treatment for cancer, with or without a solid tumour, said method of treatment will produce an effect, as measured by, for example, one or more of: the extent of the anti-tumour effect, the response rate, the time to disease progression and the survival rate.

According to the present invention there is provided a method for the production of a vascular damaging effect in a warm-blooded animal such as a human, which comprises administering to said animal an effective amount of ZD6126 or a pharmaceutically acceptable salt thereof, before, after or simultaneously with an effective amount of one of:

a) 5-FU;

b) CPT-11; and

c) 5-FU and CPT-11.

According to a further aspect of the present invention there is provided a method for the production of an anti-tumour effect in a warm-blooded animal such as a human, which comprises administering to said animal an effective amount of ZD6126 or a pharmaceutically acceptable salt thereof, before, after or simultaneously with an effective amount of one of: 5-FU; CPT-11; and 5-FU and CPT-11.

According to a further aspect of the present invention there is provided a method for the production of an anti-cancer effect in a warm-blooded animal such as a human, which comprises administering to said animal an effective amount of ZD6126 or a pharmaceutically acceptable salt thereof, before, after or simultaneously with an effective amount of one of: 5-FU; CPT-11; and 5-FU and CPT-11.

According to a further aspect of the present invention there is provided a method for the treatment of a cancer involving a solid tumour in a warm-blooded animal such as a human, which comprises administering to said animal an effective amount of ZD6126 or a pharmaceutically acceptable salt thereof, before, after or simultaneously with an effective amount of one of: 5-FU; CPT-11; and 5-FU and CPT-11.

According to a further aspect of the present invention there is provided a method for the treatment of colorectal cancer in a warm-blooded animal such as a human, which comprises administering to said animal an effective amount of ZD6126 or a pharmaceutically acceptable salt thereof, before, after or simultaneously with an effective amount of one of: 5-FU; CPT-11; and 5-FU and CPT-11.

According to a further aspect of the present invention there is provided a method for the production of a vascular damaging effect in a warm-blooded animal such as a human, which comprises administering to said animal an effective amount of ZD6126 or a pharmaceutically acceptable salt thereof, before, after or simultaneously with an effective amount of one of: 5-FU; CPT-11; and 5-FU and CPT-11; wherein ZD6126, 5-FU and CPT-11 may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of ZD6126 or a pharmaceutically acceptable salt thereof, before, after or simultaneously with an effective amount of one of: 5-FU; CPT-11; and 5-FU and CPT-11; wherein ZD6126, 5-FU and CPT-11 may each optionally be administered together with a pharmaceutically acceptable excipient or carrier.

According to a further aspect of the present invention there is provided a method for the treatment of colorectal cancer in a warm-blooded animal such as a human, which comprises administering to said animal an effective amount of ZD6126 or a pharmaceutically acceptable salt thereof, before, after or simultaneously with an effective amount of one of: 5-FU; CPT-11; and 5-FU and CPT-11; wherein ZD6126, 5-FU and CPT-11 may each optionally be administered together with a pharmaceutically acceptable excipient or carrier.

According to a further aspect of the invention there is provided a pharmaceutical composition which comprises ZD6126 or a pharmaceutically acceptable salt thereof, and 5-FU in association with a pharmaceutically acceptable excipient or carrier.

According to a further aspect of the invention there is provided a pharmaceutical composition which comprises ZD6126 or a pharmaceutically acceptable salt thereof, and CPT-11 in association with a pharmaceutically acceptable excipient or carrier.

According to a further aspect of the invention there is provided a pharmaceutical composition which comprises ZD6126 or a pharmaceutically acceptable salt thereof, and 5-FU and CPT-11 in association with a pharmaceutically acceptable excipient or carrier.

According to a further aspect of the present invention there is provided a combination product comprising ZD6126 or a pharmaceutically acceptable salt thereof and 5-FU, for use in a method of treatment of a human or animal body by therapy.

According to a further aspect of the present invention there is provided a combination product comprising ZD6126 or a pharmaceutically acceptable salt thereof and CPT-11, for use in a method of treatment of a human or animal body by therapy.

According to a further aspect of the present invention there is provided a combination product comprising ZD6126 or a pharmaceutically acceptable salt thereof and 5-FU and CPT-11, for use in a method of treatment of a human or animal body by therapy.

According to a further aspect of the present invention there is provided a combination product comprising ZD6126 or a pharmaceutically acceptable salt thereof and 5-FU, for use in a method of treatment of a human or animal body to give an anti-tumour effect.

According to a further aspect of the present invention there is provided a combination product comprising ZD6126 or a pharmaceutically acceptable salt thereof and CPT-11, for use in a method of treatment of a human or animal body to give an anti-tumour effect.

According to a further aspect of the present invention there is provided a combination product comprising ZD6126 or a pharmaceutically acceptable salt thereof and 5-FU and CPT-11, for use in a method of treatment of a human or animal body to give an anti-tumour effect.

According to a further aspect of the present invention there is provided a combination product comprising ZD6126 or a pharmaceutically acceptable salt thereof and 5-FU, for use in a method of treatment of a human or animal body to give an anti-cancer effect.

According to a further aspect of the present invention there is provided a combination product comprising ZD6126 or a pharmaceutically acceptable salt thereof and CPT-11, for use in a method of treatment of a human or animal body to give an anti-cancer effect.

According to a further aspect of the present invention there is provided a combination product comprising ZD6126 or a pharmaceutically acceptable salt thereof and 5-FU and CPT-11, for use in a method of treatment of a human or animal body to give an anti-cancer effect.

According to a further aspect of the present invention there is provided a combination product comprising ZD6126 or a pharmaceutically acceptable salt thereof and 5-FU, for use in a method of treatment of a cancer involving a solid tumour (for example, colorectal cancer) in a warm-blooded animal such as a human.

According to a further aspect of the present invention there is provided a combination product comprising ZD6126 or a pharmaceutically acceptable salt thereof and CPT-11, for use in a method of treatment of a cancer involving a solid tumour (for example, colorectal cancer) in a warm-blooded animal such as a human.

According to a further aspect of the present invention there is provided a combination product comprising ZD6126 or a pharmaceutically acceptable salt thereof and 5-FU and CPT-11, for use in a method of treatment of a cancer involving a solid tumour (for example, colorectal cancer) in a warm-blooded animal such as a human.

According to a further aspect of the present invention there is provided a kit comprising ZD6126 or a pharmaceutically acceptable salt thereof, and 5-FU.

According to a further aspect of the present invention there is provided a kit comprising ZD6126 or a pharmaceutically acceptable salt thereof, and CPT-11.

According to a further aspect of the present invention there is provided a kit comprising ZD6126 or a pharmaceutically acceptable salt thereof, and 5-FU and CPT-11.

According to a further aspect of the present invention there is provided a kit comprising:

a) ZD6126 or a pharmaceutically acceptable salt thereof in a first unit dosage form;

b) 5-FU in a second unit dosage form; and

c) container means for containing said first and second dosage forms.

According to a further aspect of the present invention there is provided a kit comprising:

a) ZD6126 or a pharmaceutically acceptable salt thereof in a first unit dosage form;

b) CPT-11 in a second unit dosage form; and

c) container means for containing said first and second dosage forms.

According to a further aspect of the present invention there is provided a kit comprising:

a) ZD6126 or a pharmaceutically acceptable salt thereof in a first unit dosage form;

b) 5-FU in a second unit dosage form;

c) CPT-11 in a third unit dosage form; and

d) container means for containing said first, second and third dosage forms.

According to a further aspect of the present invention there is provided a kit comprising:

a) ZD6126 or a pharmaceutically acceptable salt thereof, together with a pharmaceutically acceptable excipient or carrier, in a first unit dosage form;

b) 5-FU together with a pharmaceutically acceptable excipient or carrier, in a second unit dosage form; and

c) container means for containing said first and second dosage forms.

According to a further aspect of the present invention there is provided a kit comprising:

a) ZD6126 or a pharmaceutically acceptable salt thereof, together with a pharmaceutically acceptable excipient or carrier, in a first unit dosage form;

b) CPT-11 together with a pharmaceutically acceptable excipient or carrier, in a second unit dosage form; and

c) container means for containing said first and second dosage forms.

According to a further aspect of the present invention there is provided a kit comprising:

a) ZD6126 or a pharmaceutically acceptable salt thereof, together with a pharmaceutically acceptable excipient or carrier, in a first unit dosage form;

b) 5-FU together with a pharmaceutically acceptable excipient or carrier, in a second unit dosage form;

c) CPT-11 together with a pharmaceutically acceptable excipient or carrier, in a third unit dosage form; and

d) container means for containing said first, second and third dosage forms.

In an embodiment of the invention a kit as hereinbefore defined is adapted for separate, sequential or simultaneous administration of the ZD6126 and the 5-FU and/or CPT-11. Suitably in this embodiment the kit is adapted for sequential or simultaneous administration of the ZD6126 and the 5-FU and/or CPT-11.

According to a further aspect of the present invention there is provided the use of ZD6126 or a pharmaceutically acceptable salt thereof and one of:

a) 5-FU;

b) CPT-11; and

c) 5-FU and CPT-11,

in the manufacture of a medicament for use in the production of a vascular damaging effect in a warm-blooded animal such as a human.

According to a further aspect of the present invention there is provided the use of ZD6126 or a pharmaceutically acceptable salt thereof and one of:

a) 5-FU;

b) CPT-11; and

c) 5-FU and CPT-11,

in the manufacture of a medicament for use in the production of an anti-cancer effect in a warm-blooded animal such as a human.

According to a further aspect of the present invention there is provided the use of ZD6126 or a pharmaceutically acceptable salt thereof and one of:

a) 5-FU;

b) CPT-11; and

c) 5-FU and CPT-11,

in the manufacture of a medicament for use in the production of an anti-tumour effect in a warm-blooded animal such as a human.

According to a further aspect of the present invention there is provided the use of ZD6126 or a pharmaceutically acceptable salt thereof and one of:

a) 5-FU;

b) CPT-11; and

c) 5-FU and CPT-11,

in the manufacture of a medicament for use in the treatment of a cancer involving a solid tumour (for example colorectal cancer) in a warm-blooded animal such as a human.

According to a further aspect of the present invention there is provided the use of ZD6126 or a pharmaceutically acceptable salt thereof and one of:

a) 5-FU;

b) CPT-11; and

c) 5-FU and CPT-11,

in the manufacture of a medicament for use in the production of an anti-cancer effect in a warm-blooded animal such as a human wherein the cancer is a colorectal cancer.

According to a further aspect of the present invention there is provided a therapeutic combination treatment comprising the administration of an effective amount of ZD6126 or a pharmaceutically acceptable salt thereof, optionally together with a pharmaceutically acceptable excipient or carrier, and the simultaneous, sequential or separate administration of an effective amount of one of: 5-FU; CPT-11; and 5-FU and CPT-11; to a warm-blooded animal such as a human in need of such therapeutic treatment.

Such therapeutic treatment includes a vascular damaging effect, an anti-cancer effect and an anti-tumour effect.

A combination treatment of the present invention as defined herein may be achieved by way of the simultaneous, sequential or separate administration of the individual components of said treatment. A combination treatment as defined herein may be applied as a sole therapy or may involve surgery or radiotherapy or an additional chemotherapeutic agent in addition to a combination treatment of the invention. Surgery may comprise the step of partial or complete tumour resection, prior to, during or after the administration of the combination treatment with ZD6126 described herein.

Other chemotherapeutic agents for optional use with a combination treatment of the resent invention include those described in WO 99/02166 which is incorporated herein by reference. Such chemotherapy may cover five main categories of therapeutic agent:

(i) other antiangiogenic agents including vascular targeting agents;

(ii) cytostatic agents;

(iii) biological response modifiers (for example interferon);

(iv) antibodies (for example edrecolomab); and

(v) antiproliferative/antineoplastic drugs and combinations thereof, as used in medical oncology; and other categories of agent are:

(vi) antisense therapies;

(vii) gene therapy approaches; and

(ix) immunotherapy approaches.

The administration of a multiple combination of ZD6126, 5-FU and ionising radiation or ZD6126, CPT-11 and ionising radiation or ZD6126, 5-FU, CPT-11 and ionising radiation may produce effects, such as anti-tumour effects, greater than those achieved with any of ZD6126, 5-FU, CPT-11 and ionising radiation used alone. The administration of a multiple combination of ZD6126, 5-FU and ionising radiation or ZD6126, CPT-11 and ionising radiation or ZD6126, 5-FU, CPT-11 and ionising radiation may produce effects, tumour such as anti-effects, greater than those achieved with the combination of ZD6126 and 5-FU, greater than those achieved with the combination of ZD6126 and CPT-11 and greater than those achieved with the combination of ZD6126, 5-FU and CPT-11. The administration of a multiple combination of ZD6126, 5-FU and ionising radiation or ZD6126, CPT-11 and ionising radiation or ZD6126, 5-FU, CPT-11 and ionising radiation may produce effects, such as anti-tumour effects, greater than those achieved with the combination of ZD6126 and ionising radiation, greater than those achieved with the combination of 5-FU and ionising radiation, greater than those achieved with the combination of CPT-11 and ionising radiation, and greater than those achieved with the combination of 5-FU, CPT-11 and ionising radiation.

According to the present invention there is provided a method for the production of a vascular damaging effect in a warm-blooded animal such as a human, which comprises administering to said animal an effective amount of ZD6126 or a pharmaceutically acceptable salt thereof, before, after or simultaneously with an effective amount of 5-FU and before, after or simultaneously with an effective amount of ionising radiation.

According to the present invention there is provided a method for the production of a vascular damaging effect in a warm-blooded animal such as a human, which comprises administering to said animal an effective amount of ZD6126 or a pharmaceutically acceptable salt thereof, before, after or simultaneously with an effective amount of CPT-11 and before, after or simultaneously with an effective amount of ionising radiation.

According to the present invention there is provided a method for the production of a vascular damaging effect in a warm-blooded animal such as a human, which comprises administering to said animal an effective amount of ZD6126 or a pharmaceutically acceptable salt thereof, before, after or simultaneously with an effective amount of 5-FU, before, after or simultaneously with an effective amount of CPT-11 and before, after or simultaneously with an effective amount of ionising radiation.

According to the present invention there is provided a method for the treatment of a cancer in a warm-blooded animal such as a human, which comprises administering to said animal an effective amount of ZD6126 or a pharmaceutically acceptable salt thereof, before, after or simultaneously with an effective amount of 5-FU and before, after or simultaneously with an effective amount of ionising radiation.

According to the present invention there is provided a method for the treatment of a cancer in a warm-blooded animal such as a human, which comprises administering to said animal an effective amount of ZD6126 or a pharmaceutically acceptable salt thereof, before, after or simultaneously with an effective amount of CPT-11 and before, after or simultaneously with an effective amount of ionising radiation.

According to the present invention there is provided a method for the treatment of a cancer in a warm-blooded animal such as a human, which comprises administering to said animal an effective amount of ZD6126 or a pharmaceutically acceptable salt thereof, before, after or simultaneously with an effective amount of 5-FU, before, after or simultaneously with an effective amount of CPT-11 and before, after or simultaneously with an effective amount of ionising radiation.

According to the present invention there is provided a method for the treatment of a cancer involving a solid tumour in a warm-blooded animal such as a human, which comprises administering to said animal an effective amount of ZD6126 or a pharmaceutically acceptable salt thereof, before, after or simultaneously with an effective amount of 5-FU and before, after or simultaneously with an effective amount of ionising radiation.

According to the present invention there is provided a method for the treatment of a cancer involving a solid tumour in a warm-blooded animal such as a human, which comprises administering to said animal an effective amount of ZD6126 or a pharmaceutically acceptable salt thereof, before, after or simultaneously with an effective amount of CPT-11 and before, after or simultaneously with an effective amount of ionising radiation.

According to the present invention there is provided a method for the treatment of a cancer involving a solid tumour in a warm-blooded animal such as a human, which comprises administering to said animal an effective amount of ZD6126 or a pharmaceutically acceptable salt thereof, before, after or simultaneously with an effective amount of 5-FU, before, after or simultaneously with an effective amount of CPT-11 and before, after or simultaneously with an effective amount of ionising radiation.

According to a further aspect of the present invention there is provided a method for the production of a vascular damaging effect in a warm-blooded animal such as a human, which comprises administering to said animal an effective amount of ZD6126 or a pharmaceutically acceptable salt thereof, before, after or simultaneously with an effective amount of 5-FU and before, after or simultaneously with an effective amount of ionising radiation, wherein ZD6126 and 5-FU may each optionally be administered together with a pharmaceutically acceptable excipient or carrier.

According to a further aspect of the present invention there is provided a method for the production of a vascular damaging effect in a warm-blooded animal such as a human, which comprises administering to said animal an effective amount of ZD6126 or a pharmaceutically acceptable salt thereof, before, after or simultaneously with an effective amount of CPT-11 and before, after or simultaneously with an effective amount of ionising radiation, wherein ZD6126 and CPT-11 may each optionally be administered together with a pharmaceutically acceptable excipient or carrier.

According to a further aspect of the present invention there is provided a method for the production of a vascular damaging effect in a warm-blooded animal such as a human, which comprises administering to said animal an effective amount of ZD6126 or a pharmaceutically acceptable salt thereof, before, after or simultaneously with an effective amount of 5-FU, before, after or simultaneously with an effective amount of CPT-11 and before, after or simultaneously with an effective amount of ionising radiation, wherein ZD6126, 5-FU and CPT-11 may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of ZD6126 or a pharmaceutically acceptable salt thereof, before, after or simultaneously with an effective amount of 5-FU and before, after or simultaneously with an effective amount of ionising radiation, wherein ZD6126 and 5-FU may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of ZD6126 or a pharmaceutically acceptable salt thereof, before, after or simultaneously with an effective amount of CPT-11 and before, after or simultaneously with an effective amount of ionising radiation, wherein ZD6126 and CPT-11 may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of ZD6126 or a pharmaceutically acceptable salt thereof, before, after or simultaneously with an effective amount of 5-FU, before, after or simultaneously with an effective amount of CPT-11 and before, after or simultaneously with an effective amount of ionising radiation, wherein ZD6126, 5-FU and CPT-11 may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of ZD6126 or a pharmaceutically acceptable salt thereof, before, after or simultaneously with an effective amount of 5-FU and before, after or simultaneously with an effective amount of ionising radiation, wherein ZD6126 and 5-FU may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of ZD6126 or a pharmaceutically acceptable salt thereof, before, after or simultaneously with an effective amount of CPT-11 and before, after or simultaneously with an effective amount of ionising radiation, wherein ZD6126 and CPT-11 may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of ZD6126 or a pharmaceutically acceptable salt thereof, before, after or simultaneously with an effective amount of 5-FU, before, after or simultaneously with an effective amount of CPT-11 and before, after or simultaneously with an effective amount of ionising radiation, wherein ZD6126, 5-FU and CPT-11 may each optionally be administered together with a pharmaceutically acceptable excipient or carrier.

According to a further aspect of the present invention there is provided the use of ZD6126 or a pharmaceutically acceptable salt thereof and 5-FU in the manufacture of a medicament for use in the production of a vascular damaging effect in a warm-blooded animal such as a human which is being treated with ionising radiation.

According to a further aspect of the present invention there is provided the use of ZD6126 or a pharmaceutically acceptable salt thereof and CPT-11 in the manufacture of a medicament for use in the production of a vascular damaging effect in a warm-blooded animal such as a human which is being treated with ionising radiation.

According to a further aspect of the present invention there is provided the use of ZD6126 or a pharmaceutically acceptable salt thereof and 5-FU and CPT-11 in the manufacture of a medicament for use in the production of a vascular damaging effect in a warm-blooded animal such as a human which is being treated with ionising radiation.

According to a further aspect of the present invention there is provided the use of ZD6126 or a pharmaceutically acceptable salt thereof and 5-FU in the manufacture of a medicament for use in the production of an anti-cancer effect in a warm-blooded animal such as a human which is being treated with ionising radiation.

According to a further aspect of the present invention there is provided the use of ZD6126 or a pharmaceutically acceptable salt thereof and CPT-11 in the manufacture of a medicament for use in the production of an anti-cancer effect in a warm-blooded animal such as a human which is being treated with ionising radiation.

According to a further aspect of the present invention there is provided the use of ZD6126 or a pharmaceutically acceptable salt thereof and 5-FU and CPT-11 in the manufacture of a medicament for use in the production of an anti-cancer effect in a warm-blooded animal such as a human which is being treated with ionising radiation.

According to a further aspect of the present invention there is provided the use of ZD6126 or a pharmaceutically acceptable salt thereof and 5-FU in the manufacture of a medicament for use in the production of an anti-tumour effect in a warm-blooded animal such as a human which is being treated with ionising radiation.

According to a further aspect of the present invention there is provided the use of ZD6126 or a pharmaceutically acceptable salt thereof and CPT-11 in the manufacture of a medicament for use in the production of an anti-tumour effect in a warm-blooded animal such as a human which is being treated with ionising radiation.

According to a further aspect of the present invention there is provided the use of ZD6126 or a pharmaceutically acceptable salt thereof and 5-FU and CPT-11 in the manufacture of a medicament for use in the production of an anti-tumour effect in a warm-blooded animal such as a human which is being treated with ionising radiation.

According to a further aspect of the present invention there is provided a therapeutic combination treatment comprising the administration of an effective amount of ZD6126 or a pharmaceutically acceptable salt thereof, optionally together with a pharmaceutically acceptable excipient or carrier, and the administration of an effective amount of 5-FU, optionally together with a pharmaceutically acceptable excipient or carrier and the administration of an effective amount of ionising radiation, to a warm-blooded animal such as a human in need of such therapeutic treatment wherein the ZD6126, 5-FU and ionising radiation may be administered simultaneously, sequentially or separately and in any order.

According to a further aspect of the present invention there is provided a therapeutic combination treatment comprising the administration of an effective amount of ZD6126 or a pharmaceutically acceptable salt thereof, optionally together with a pharmaceutically acceptable excipient or carrier, and the administration of an effective amount of CPT-11, optionally together with a pharmaceutically acceptable excipient or carrier and the administration of an effective amount of ionising radiation, to a warm-blooded animal such as a human in need of such therapeutic treatment wherein the ZD6126, CPT-11 and ionising radiation may be administered simultaneously, sequentially or separately and in any order.

According to a further aspect of the present invention there is provided a therapeutic combination treatment comprising the administration of an effective amount of ZD6126 or a pharmaceutically acceptable salt thereof, optionally together with a pharmaceutically acceptable excipient or carrier, and the administration of an effective amount of 5-FU, optionally together with a pharmaceutically acceptable excipient or carrier, and the administration of an effective amount of CPT-11, optionally together with a pharmaceutically acceptable excipient or carrier, and the administration of an effective amount of ionising radiation, to a warm-blooded animal such as a human in need of such therapeutic treatment wherein the ZD6126, 5-FU, CPT-11 and ionising radiation may be administered simultaneously, sequentially or separately and in any order.

A warm-blooded animal such as a human which is being treated with ionising radiation means a warm-blooded animal such as a human which is treated with ionising radiation before, after or at the same time as the administration of a medicament or combination treatment comprising ZD6126 and one of: 5-FU; CPT-11; and 5-FU and CPT-11. For example said ionising radiation may be given to said warm-blooded animal such as a human within the period of a week before to a week after the administration of a medicament or combination treatment comprising ZD6126 and one of: 5-FU; CPT-11; and 5-FU and CPT-11. This means that ZD6126, 5-FU, CPT-11 and ionising radiation may be administered separately or sequentially in any order, or may be administered simultaneously. The warm-blooded animal may experience the effect of each of ZD6126, 5-FU, CPT-11 and radiation simultaneously.

According to one aspect of the present invention the ionising radiation is administered before one of ZD6126 and one of: 5-FU; CPT-11; and 5-FU and CPT-11, or after one of ZD6126 and one of: 5-FU; CPT-11; and 5-FU and CPT-11.

According to one aspect of the present invention the ionising radiation is administered before any of ZD6126 and one of: 5-FU; CPT-11; and 5-FU and CPT-11 or after all of ZD6126 and one of: 5-FU; CPT-11; and 5-FU and CPT-11.

According to another aspect of the present invention ZD6126 is given after the other element(s) of the combination treatment.

As stated above the combination treatments, uses, compositions and kits of the present invention as defined herein are of interest for their vascular damaging effects. The term “vascular damaging effect” refers to damage to neovasculature that has formed as a result of inappropriate angiogenesis (i.e. pathological angiogenesis). The damage to such neovasculature may result in loss of structure of the neovasculature, reduced blood flow through the neovasculature, or leakage of blood from the neovasculature. Accordingly, in a tumour the vascular damaging effect of the vascular targeting agent on the neovasculature supplying blood to the tumour. The vascular damaging effect on, for example a tumour, includes reduction in blood flow to the tumour and/or the degree of tumour necrosis and/or a reduction in tumour size. The extent of vascular damage produced by a vascular damaging agent can be measured using known techniques, for example by suitable imaging methods known in the art, for example CT scanning or MRI (Dowlati et al., Cancer Research 62, 3408-3416, June 2002), or PET (Anderson et al., Journal of Clinical Oncology 21, 2823-2830, August 2003).

The combination treatments, uses, compositions and kits according to the invention are expected to be useful in the prophylaxis and treatment of a wide range of disease states where inappropriate angiogenesis occurs including cancer, (including leukaemia, multiple myeloma and lymphoma), diabetes, psoriasis, rheumatoid arthritis, Kaposi's sarcoma, haemangioma, acute and chronic nephropathies, atheroma, arterial restenosis, autoimmune diseases, acute inflammation, endometriosis, dysfunctional uterine bleeding and ocular diseases with retinal vessel proliferation including age-related macular degeneration. In particular such combination treatments of the invention are expected to slow advantageously the growth of primary and recurrent solid tumours of, for example, the colon, breast, prostate, lungs and skin.

According to one aspect of the present invention such combination treatments, uses, compositions and kits of the invention are expected to slow advantageously the growth of primary and secondary (recurrent) tumours in colorectal cancer.

According to another aspect of the present invention the effect of a method of treatment or use of the present invention is expected to be at least equivalent to the addition of the effects of each of the components of said treatment used alone, that is, of each of ZD6126, CPT-11, 5-FU and ionising radiation used alone.

According to another aspect of the present invention the effect of a method of treatment or use of the present invention is expected to be greater than the addition of the effects of each of the components of said treatment used alone, that is, of each of ZD6126, CPT-11, 5-FU and ionising radiation, used alone.

According to another aspect of the present invention the effect of a method of treatment or use of the present invention is expected to be a synergistic effect.

According to the present invention a combination treatment or use according to the invention is defined as affording a synergistic effect if the effect is therapeutically superior, as measured by, for example, the extent of the response, the response rate, the time to disease progression or the survival period, to that achievable on dosing one or other of the components of the combination treatment at its conventional dose. For example, the effect of a combination treatment is synergistic if the effect is therapeutically superior to the effect achievable with ZD6126, 5-FU, CPT-11, 5-FU and CPT-11, or ionising radiation used alone. Further, the effect of the combination treatment is synergistic if a beneficial effect is obtained in a group of patients that does not respond (or responds poorly) to ZD6126, 5-FU, CPT-11, 5-FU and CPT-11, or ionising radiation used alone. In addition, the effect of a combination treatment is defined as affording a synergistic effect if one of the components is dosed at its conventional dose and the other component(s) is/are dosed at a reduced dose and the therapeutic effect, as measured by, for example, the extent of the response, the response rate, the time to disease progression or the survival period, is equivalent to that achievable on dosing conventional amounts of the components of the combination treatment. In particular, synergy is deemed to be present if the conventional dose of ZD6126, 5-FU, CPT-11, 5-FU and CPT-11, or ionising radiation may be reduced without detriment to one or more of the extent of the response, the response rate, the time to disease progression and survival data, in particular without detriment to the duration of the response, but with fewer and/or less troublesome side-effects than those that occur when conventional doses of each component are used.

The ZD6126, 5-FU and CPT-11 used in the methods, uses and kits according to the present invention may be administered by way of the simultaneous, sequential or separate administration of the individual components as described hereinbefore. The term “simultaneous administration” used herein refers to the administration to a patient of one or more components of the invention at approximately the same time. For example ZD6126 may be administered as an IV dose at the same time as an IV dose of 5-FU. The term “sequential administration” refers to administration of one component of a treatment followed shortly after by administration of one or more of the remaining components. For example ZD6126 may be administered as an IV dose followed, a short time later by an IV infusion of 5-FU and/or CPT-11. Suitably the second component of the treatment is administered less than 4 hours, preferably less than 3 hours, more preferably less than 1 hour following administration of the first component of the treatment. The term “separate administration” refers to administration of one component of the treatment after the administration of another component. As will be understood, when separate administration is used, the second component of a treatment should be administered before the therapeutic effect, for example an anti-tumour effect, of the first component has been lost. For example when ZD6126 is administered as a first component of a treatment, the second component such as 5-FU should be administered before the anti-tumour effect of the ZD6126 (such as the anti-tumour effects described hereinbefore for example tumour necrosis, tumour shrinkage or inhibition of tumour growth) has been lost. Accordingly, for separate administration the time between doses of each component will generally be less than 2 months, more particularly less than 1 month, still more particularly less than 2 weeks, for example less than 1 week. For example the time between doses of each component may be from 4 hours to 1 month, particularly, 6 hours to 2 weeks, more particularly 12 hours to 1 week, or 24 hours to 1 week are suitable for separate administration of the components of the treatment. As mentioned hereinbefore, each component of the combination may be administered in any order. The route of administration may be the same or different for each component of the treatment as described herein after. Accordingly, each component may be administered parenterally such as by IV dosing, or one component may be administered orally and one parenterally.

The compositions described herein may be in a form suitable for oral administration, for example as a tablet or capsule, for parenteral injection (including intravenous, subcutaneous, intramuscular, intravascular or infusion) for example as a sterile solution, suspension or emulsion, for topical administration for example as an ointment or cream, for rectal administration for example as a suppository or the route of administration may be by direct injection into the tumour or by regional delivery or by local delivery. In other embodiments of the present invention the ZD6126 of the combination treatment may be delivered endoscopically, intratracheally, intralesionally, percutaneously, intravenously, subcutaneously, intraperitoneally or intratumourally. Preferably ZD6126 is administered intravenously. In general the compositions described herein may be prepared in a conventional manner using conventional excipients. The compositions of the present invention are advantageously presented in unit dosage form.

ZD6126 will normally be administered to a warm-blooded animal at a unit dose within the range 10-500 mg per square metre body area of the animal, for example approximately 0.3-15 mg/kg in a human. A unit dose in the range, for example, 0.3-15 mg/kg, preferably 0.5-5 mg/kg is envisaged and this is normally a therapeutically-effective dose. A unit dosage form such as a tablet or capsule will usually contain, for example 25-250 mg of active ingredient. Preferably a daily dose in the range of 0.5-5 mg/kg is employed.

It has been reported, in International Patent Application Publication No. WO 01/74369, that the effect of a given dose of ZD6126 can be increased by administering it in divided doses. Divided doses, also called split doses, means that the total dose to be administered to a warm-blooded animal, such as a human, in any one day period (for example one 24 hour period from midnight to midnight) is divided up into two or more fractions of the total dose (for example 2, 3, 4 or 5 doses) and these fractions are administered with a time period between each fraction of about greater than 0 hours to about 10 hours, preferably about 1 hour to about 6 hours, more preferably about 2 hours to about 4 hours. The fractions of total dose may be about equal or unequal.

For example the total dose may be divided into two parts which may be about equal with a time interval between doses of greater than or equal to two hours and less than or equal to 6 hours, more particularly with a time interval between doses of greater than or equal to two hours and less than or equal to 4 hours.

ZD6126 may be administered in divided doses when used in combination with one of: 5-FU; CPT-11; and 5-FU and CPT-11; and optionally with ionising radiation.

CPT-11 is also known as irinotecan. CPT-11 may be administered in accordance with any known route of administration and dosage.

For example CPT-11 may be dosed at 350 mg/m² as an intravenous infusion over a 30 to 90 minute period every 3 weeks.

5-FU is 5-fluorouracil. 5-FU may be administered according to any known route of administration and dosage.

For example 5-FU may be given as an intravenous daily infusion of 15 mg/kg diluted in 500 ml of 5% dextrose solution or 500 ml 0.9% sodium chloride solution given by intravenous infusion: at the rate of 40 drops per minute over 4 hours; or infused over 30 to 60 minutes; or as a daily continuous infusion over 24 hours. The daily dose of 5-FU is recommended not to exceed 1 g. 5-FU is usually given daily in one of these ways until 12-15 g has been given and this constitutes one course of 5-FU. It is usual practice to leave 4 to 6 weeks between courses of 5-FU. Alternatively 5-FU may be dosed by intravenous injection at a dose of 12 mg/kg on three consecutive days, followed by 6 mg/kg on days 5, 7 and 9 i.e. on the three following alternate days, followed by a maintenance dose of 5-15 mg/kg by intravenous injection once a week. Alternatively 5-FU may be given by intravenous injection at a dose of 15 mg/kg once a week for the duration of the patient's treatment. 5-FU may also be dosed intra-arterially as a regional perfusion at 5-7.5 mg/kg by 24 hour continuous infusion. 5-FU may also be dosed orally at a dose of 15 mg/kg once a week or at a dose of 15 mg/kg for six successive days followed by 15 mg/kg once a week.

5-FU is commonly administered with leucovorin. For the avoidance of doubt the combination treatments of the present invention include the use of 5-FU when given with, or without, leucovorin.

Leucovorin may be administered according to any known route of administration and dosage. When used in combination with 5-FU, leucovorin (folinic acid) is conveniently administered as calcium leucovorin and given intravenously. For example, calcium leucovorin may be given at a dose of 200 mg/m² by slow intravenous injection, followed immediately by 5-FU at an initial dose of 370 mg/m² by intravenous injection. The injection of leucovorin should not be given more rapidly than over 3-5 minutes because of the calcium content of the solution. This treatment is repeated daily for 5 consecutive days. Subsequent courses may be given after a treatment-free interval of 21-28 days.

Alternatively the following regimen may be used: leucovorin 500 mg/m² given by 2 hour infusion every week for 6 weeks with 5-FU 500 mg/m² given as an iv bolus midway through the 6-week period.

Alternatively the following regimen may be used: leucovorin 200 mg/m² given by iv 2 hour infusion followed by 5-FU 400 mg/m² iv bolus followed by 5-FU 600 mg/m² given by iv 22 hour infusion, repeated for 2 consecutive days. The cycle is repeated every 2 weeks.

Alternatively 5-FU may be administered orally as capecitabine (Xeloda™), tegafur (particularly in combination with uracil), or TS-1. Capecitabine is a relatively non-cytotoxic fluoropyrimidine carbamate which functions as an orally administered precursor of 5-FU. Capecitabine may be administered according to any known dosage. For example a dose of 1250 mg/m² may be given orally twice a day, (equivalent to a daily dose of 2500 mg/m²), for 14 days followed by a rest period of 7 days.

Combination treatments of the present invention include the use of 5-FU when given in any form (including prodrug and precursor forms that are converted to 5-FU systemically or within the tumour), when administered via any route and when given with, or without, leucovorin.

Radiotherapy may be administered according to the known practices in clinical radiotherapy. The dosages of ionising radiation will be those known for use in clinical radiotherapy. The radiation therapy used will include for example the use of γ-rays, X-rays, and/or the directed delivery of radiation from radioisotopes. Other forms of DNA damaging factors are also included in the present invention such as microwaves and UV-irradiation. For example X-rays may be dosed in daily doses of 1.8-2.0 Gy, 5 days a week for 5-6 weeks. Normally a total fractionated dose will lie in the range 45-60 Gy. Single larger doses, for example 5-10 Gy may be administered as part of a course of radiotherapy. Single doses may be administered intraoperatively. Hyperfractionated radiotherapy may be used whereby small doses of X-rays are administered regularly over a period of time, for example 0.1 Gy per hour over a number of days. Dosage ranges for radioisotopes vary widely, and depend on the half-life of the isotope, the strength and type of radiation emitted, and on the uptake by cells.

As stated above the size of the dose of each therapy which is required for the therapeutic or prophylactic treatment of a particular disease state will necessarily be varied depending on the host treated, the route of administration and the severity of the illness being treated. Accordingly the optimum dosage may be determined by the practitioner who is treating any particular patient. For example, it may be necessary or desirable to reduce the above-mentioned doses of the components of the combination treatments in order to reduce toxicity.

The combination treatments of the present invention comprise: ZD6126 and 5-FU; ZD6126 and CPT-11; ZD6126, 5-FU and CPT-11; ZD6126, 5-FU and ionising radiation; ZD6126, CPT-11 and ionising radiation; ZD6126, 5-FU, CPT-11 and ionising radiation. The agents therein may be administered separately or sequentially in any order, or may be administered simultaneously. As stated hereinbefore, for each combination treatment 5-FU may be administered with or without leucovorin. Ionising radiation, when used, may also be administered separately or sequentially in any order, or may be administered simultaneously with, the ZD6126, 5-FU and/or CPT-11.

The present invention relates to combinations of ZD6126 or a salt thereof with one of 5-FU; CPT-11; 5-FU and CPT-11. Salts of ZD6126 for use in pharmaceutical compositions will be pharmaceutically acceptable salts, but other salts may be useful in the production of ZD6126 and its pharmaceutically acceptable salts. Such salts may be formed with an inorganic or organic base which affords a pharmaceutically acceptable cation. Such salts with 30 inorganic or organic bases include for example an alkali metal salt, such as a sodium or potassium salt, an alkaline earth metal salt such as a calcium or magnesium salt, an ammonium salt or for example a salt with methylamine, dimethylamine, trimethylamine, piperidine, morpholine or tris-(2-hydroxyethyl)amine.

According to WO 99/02166, ZD6126 may be made according to the following process.

N-Acetylcolchinol (30.0 g, 83.9 mmol) is dissolved in acetonitrile under an inert atmosphere and 1,2,3-triazole (14.67 g, 212.4 mmol) added via a syringe. Di-tert-butyl-diethylphosphoramidite (37.7 g, 151.4 mmol) is added and the reaction mixture stirred at about 20° C. to complete the formation of the intermediate phosphite ester. Cumene hydroperoxide (24.4 g, 159.2 mmol) is added at about 10° C. and the reaction mixture stirred until the oxidation is complete. Butyl acetate (50 ml) and sodium hydroxide solution (250 ml of 1M) are added, the reaction mixture stirred and the aqueous phase discarded. The organic solution is washed with sodium hydroxide solution (2×250 ml of 1M) and a saturated solution of sodium chloride. Trifluoroacetic acid (95.3 g, 836 mmol) is added at about 15° C. The reaction mixture is distilled at atmospheric pressure, ZD6126 crystallises and is isolated at ambient temperature.

The following tests were used to demonstrate the activity of ZD6126 in combination with 5-FU and CPT-11.

Human LS-174T Colon Tumour Xenografts in Nude Mice

1×10⁷ LS-174T human colorectal tumour cells were inoculated subcutaneously in 0.2 ml of RPMI medium into the right flank of Nude mice.

The treatments began when the mean tumour volume reached approximately 400 mm³ (Day 1). Mice were randomised into 6 groups of 15-18 mice per group. The treatment schedule is summarized in Table 1.

Group 1—control

Group 2—received 3 weekly intraperitoneal (IP) injections of ZD6126 at 200 mg/kg on Days 1, 8 and 15

Group 3—received 3 weekly intravenous (IV) injections of 5-FU at 75 mg/kg on Days 1, 8 and 15

Group 4—received 3 weekly IV injections of CPT-11 alone at 25 mg/kg on Days 1, 8 and 15.

Group 5—received 3 weekly IP injections of ZD6126 at 200 mg/kg on Days 1, 8 and 15 and 3 weekly IV injections of 5-FU at 75 mg/kg Days 1, 8 and 15.

Group 6—received 3 weekly IP injections of ZD6126 at 200 mg/kg on Days 1, 8 and 15 and 3 weekly IV injections of CPT-11 at 25 mg/kg on Days 1, 8 and 15.

In groups 5 and 6, when animals received both ZD6126 and CPT-11 or 5-FU on the same day, the ZD6126 was administered 2 hours after the CPT-11 or 5-FU.

TABLE 1

| | | | | | DAYS-INTERVAL |
|---|---|---|---|---|---|
| | | | DOSES | | BETWEEN |
| GROUPS | NO. MICE | TREATMENTS | (MG/KG) | ROUTE | TREATMENTS |
| 1 | 18 | Vehicle of ZD6126 | NA | IP | Weekly ×3 |
| | | Vehicle of 5-FU | NA | IV | Weekly ×3 |
| | | and CPT-11 | | | |
| 2 | 18 | ZD6126 | 200.0 | IP | Weekly ×3 |
| 3 | 15 | 5-FU | 75.0 | IV | Weekly ×3 |
| 4 | 15 | CPT-11 | 25.0 | IV | Weekly ×3 |
| 5 | 18 | ZD6126 | 200 | IP | Weekly ×3 |
| | | 5-FU | 75 | IV | Weekly ×3 |
| 6 | 18 | ZD6126 | 200 | IP | Weekly ×3 |
| | | CPT-11 | 25 | IV | Weekly ×3 |

NA = not applicable

For all animals, the tumour size was measured twice a week with calipers, and the tumour volume (mm³) was estimated according to the formula: {[(Square root of Length×Width)×(Length×Width)]×0.5236}. The mice were sacrificed when tumours grew beyond 10% of bodyweight.

Treatment efficacy was assessed in terms of the effects on the tumour volumes of treated mice relative to vehicle-treated mice and expressed as growth delay. Growth delay T-(C) was calculated as the difference in the median growth time of the treatment group (T) and the vehicle control group (C) to reach a tumor size of 2 cm³.

TABLE 2
Results
Effect of treatments on the growth of SC LS-174T tumours
xenografted in Nude mice treated with ZD6126 alone or in
combination with 5-FU or CPT-11.

| | | | | MEDIAN TIME | |
|---|---|---|---|---|---|
| | | | | TO REACH | |
| | | | | TUMOUR SIZE | |
| | | DOSES | | OF 2 CM³ | T − C |
| GROUPS | TREATMENTS | (MG/KG/) | ROUTE | (DAYS) | (DAYS) |
| 1 | Vehicle of ZD6126 | 0.0 | IP | 9 | NA |
| | Vehicle of 5-FU | | IV | | |
| | and CPT-11 | | | | |
| 2 | ZD6126 | 200.0 | IP | 17 | 8 |
| 3 | 5-FU | 75.0 | IV | 14 | 5 |
| 4 | CPT-11 | 25.0 | IV | 17 | 8 |
| 5 | ZD6126 | 200 | IP | 22 | 13 |
| | 5-FU | 75 | IV | | |
| 6 | ZD6126 | 200 | IP | 22 | 13 |
| | CPT-11 | 25 | IV | | |

Treatment with ZD6126, 5-FU or CPT-11 induced tumour growth delays (I-C) of 8 days, 5 days and 8 days respectively, when compared with control tumours. The combination of ZD6126 and 5-FU induced a tumour growth delay (T-C) of 13 days, which was greater than the growth delay induced by either ZD6126 or 5-FU alone. The combination of ZD6126 and CPT-11 induced a tumour growth (T-C) of 13 days, which was greater than the growth delay induced by either CPT-11 or ZD6126 alone.

In the LS174T mouse xenograft model of human colorectal cancer, ZD6126 combined with either 5-FU or CPT-11 induces greater tumour growth delay than any of the single agents alone.

An analogous experiment may be used to look at the combination of ZD6126, CPT-11 and 5-FU in this animal model.

1. Use of ZD6126 or a pharmaceutically acceptable salt thereof and one of:
a) 5-FU;
b) CPT-11; and
c) 5-FU and CPIT-11,
in the manufacture of a medicament for use in the production of a vascular damaging effect in a warm-blooded animal.

2. Use according to claim 1 for use in the production of an anti-tumour effect in a warm-blooded animal.

3. Use according to claim 1 for use in the production of an anti-cancer effect in a warm-blooded animal.

4. Use according to claim 1 for use in the treatment of a cancer involving a solid tumour in a warm-blooded animal.

5. Use according to claim 4 wherein the cancer is colorectal cancer.

6. Use according to any one of the preceding claims wherein the ZD6126 or a pharmaceutically acceptable salt thereof is used with 5-FU.

7. Use according to any one of claims 1 to 5 wherein the ZD6126 or a pharmaceutically acceptable salt thereof is used with CPT-11.

8. Use according to any one of claims 1 to 5 wherein the ZD6126 or a pharmaceutically acceptable salt thereof is used with 5-FU and CPT-11.

9. Use according to any one of the preceding claims wherein the warm-blooded animal is being treated with ionising radiation.

10. Use according to any one of the preceding claims wherein the ZD6126 or a pharmaceutically acceptable salt thereof is administered to the warm-blooded animal in divided doses.

11. Use according to any one of claims 1 to 6 wherein the 5-FU is administered to the warm-blooded animal in the form of a prodrug of 5-FU.

12. A pharmaceutical composition comprising ZD6126 or a pharmaceutically acceptable salt thereof, and 5-FU in association with a pharmaceutically acceptable excipient or carrier.

13. A pharmaceutical composition comprising ZD6126 or a pharmaceutically acceptable salt thereof, and CPIT-11 in association with a pharmaceutically acceptable excipient or carrier.

14. A pharmaceutical composition comprising ZD6126 or a pharmaceutically acceptable salt thereof, and 5-FU and CPT-11 in association with a pharmaceutically acceptable excipient or carrier.

15. A kit comprising ZD6126 or a pharmaceutically acceptable salt thereof, and 5-FU.

16. A kit comprising ZD6126 or a pharmaceutically acceptable salt thereof, and CPT-11.

17. A kit comprising ZD6126 or a pharmaceutically acceptable salt thereof, and 5-FU and CPT-11.

18. A method for the production of a vascular damaging effect in a warm-blooded animal such as a human, which comprises administering to said animal an effective amount of ZD6126 or a pharmaceutically acceptable salt thereof, before, after or simultaneously with an effective amount of one of:
a) 5-FU;
b) CPT-11; and
c) 5-FU and CPT-11.

19. A method for the treatment of a cancer involving a solid tumour in a warm-blooded animal such as a human, which comprises administering to said animal an effective amount of ZD6126 or a pharmaceutically acceptable salt thereof, before, after or simultaneously with an effective amount of one of:
a) 5-FU;
b) CPT-11; and
c) 5-FU and CPT-11.

20. A method according to claim 19 wherein the cancer is colorectal cancer.

21. A method according to any one of claims 18 to 20 wherein the ZD6126 or a pharmaceutically acceptable salt thereof is administered before, after or simultaneously with an effective amount of 5-FU.

22. A method according to any one of claims 18 to 20 wherein ZD6126 or a pharmaceutically acceptable salt thereof is administered before, after or simultaneously with an effective amount of CPT-11.

23. A method according to any one of claims 18 to 20 wherein ZD6126 or a pharmaceutically acceptable salt thereof is administered before, after or simultaneously with an effective amount of 5-FU, and before, after or simultaneously with an effective amount of CPT-11.

24. A method according to any one claims 18 to 20 further comprising administration to said warm-blooded animal an effective amount of ionising radiation, wherein the ZD6126, ionising radiation, 5-FU and/or CPT-11 are administered simultaneously, sequentially or separately in any order.

25. A method according to any one of claims 18 to 20 wherein the ZD6126 or a pharmaceutically acceptable salt thereof, is administered to said warm-blooded animal in divided doses.

26. A method according to any one of claims 18 to 21 wherein the 5-FU is administered to said warm-blooded animal in the form of a prodrug of 5-FU.
